(19) United States
(12) Patent Application Publication
(10) Pub. No.: US 2018/0071326 A1
(43) Pub. Date: Mar. 15, 2018

(54) METHODS TO PREVENT TISSUE COLONIZATION OF PATHOGENS AND FOR TREATMENT OF BIOFILM ON ANIMAL TISSUES, INCLUDING TREATMENT OF INFECTION

(71) Applicant: Reduxx LLC, Fort Wayne, IN (US)

(72) Inventors: Carl Hilliard, San Diego, CA (US); William R. Cast, Fort Wayne, IN (US)

(73) Assignee: Reduxx LLC, Fort Wayne, IN (US)

(21) Appl. No.: 15/818,553

(22) Filed: Nov. 20, 2017

Related U.S. Application Data
(63) Continuation of application No. PCT/US2016/033208, filed on May 19, 2016.
Application No. PCT/US2016/033208 is a continuation-in-part of application No. 14/716,589, filed on May 19, 2015.
Application No. 14/716,589 is a continuation-in-part of application No. 14/716,566, filed on May 19, 2015, now abandoned.

(51) Int. Cl. A61K 31/695; A61K 9/50; A61K 9/00; A61K 9/12; A01N 55/00; A01N 25/28; A61K 45/06

(52) U.S. Cl. CPC A61K 31/695; A61K 9/5015; A61K 9/0046; A61K 45/06; A61K 9/0017; A01N 55/00; A01N 25/28; A61K 9/12

(57) ABSTRACT

An antimicrobial product and methods of use are provided. The antimicrobial product includes a water-soluble antimicrobial organosilane (3-(trihydroxysilyl) propyldimethyloctadecyl ammonium chloride) and various additional adjuvant compounds, including anti-inflammatory medications, antiseptics, penetrants and/or detergents. An example delivery system including a microcapsule encasing the product is also described. Methods of use include embedding a delivery system within an article, such as bandages, inserts or wound coverings, the microcapsule containing the product which is released by a mechanism and at a time particular to the intended use of the article. The methods include topically treating a biofilm infection on an animal tissue substrate by disrupting existing microbial biofilm growth, penetrating existing biofilms, thereby killing microbes, and also through providing residual bonded organosilane, then killing persister cell microbes that through degrees of dormancy have escaped being killed.

### CROSS-REFERENCE TO RELATED APPLICATIONS

This application is a continuation of P.C.T. Application No. PCT/US2016/033208, filed May 19, 2016, which is a continuation-in-part and claims the benefit of U.S. application Ser. No. 14/716,589, filed May 19, 2015, and U.S. application Ser. No. 14/716,566, filed May 19, 2015. The entirety of these applications are hereby incorporated by reference for all purposes.

### BACKGROUND

### Technical Field

This disclosure relates generally to antimicrobial compounds with antimicrobial activity; in particular, to organosilane quaternary ammonium compounds for topical treatment of biofilms and other microbial pathogens in animals and humans, including eyes, ear canals, skin and sub-cutis, toe nails and infected wounds and abrasions.

### State of the Art

Prevention and treatment of infection in humans and animals has been a public health goal since the discovery of microorganisms and their role in causing disease. As an outgrowth of the germ theory of disease, much progress has been made in controlling the spread, dissemination, and effects of pathogenic microorganisms. For example, simple techniques such as routine hand-washing and thorough cleaning of hard surfaces are highly effective in preventing the spread of diseases which are disseminated by contact. One such method is by the use of face masks that are designed to filter out, and, in some cases, destroy microorganisms by use of chemicals embedded in the mask. Some masks are used for a short duration, for example surgical masks. Other masks are worn by the general population for longer periods, usually when the wearer is ill for the duration of a respiratory infection. The disadvantages of these methods is that either live pathogens remain embedded in the used face mask or the mask contains chemicals that are harmful to the environment when disposed. Furthermore, the presence of toxic biocides in the environment may lead to mutations in microorganisms that then become resistant to such biocides. When infection occurs despite such precautions, treatment with topical and systemic antimicrobials, such as the use of antibiotics, have been valuable adjuncts to these preventive measures. It is widely perceived that antiseptics and disinfectants act as general protoplasmic poisons. Antibiotics generally act using one of four mechanisms: “Inhibition or regulation of enzymes involved in cell wall biosynthesis, nucleic acid metabolism and repair, or protein synthesis, or “disruption of the membrane structure.” “Antibiotics that affect the structure of the cell wall act at different stages of peptidoglycan synthesis and cell wall construction.”

These topical medications are losing effectiveness and microbial infections remain the number one cause of death globally. WHO has called antimicrobial resistance “so serious that it threatens the achievements of modern medicine.” An important reason for antibiotic failure is biofilm formation. A biofilm may be formed by particles, organic or inorganic, that flow or settle onto the surface, including layers of dead microorganisms, their products and detritus “the Conditioning Layer.” The Conditioning Layer facilitates the adhesion of planktonic microbes to the animal tissue substrate and provides nutrients and proximate pathogens that aid in the growth and defense of the colony. Upon attachment to the Conditioning Layer, microbes begin to divide into new cells and to produce EPS (Extracellular Polymeric Substance) and the three dimensional biofilm begins to take shape, the EPS aiding in adherence. Inter-cellular communications are established between the cells within the biofilm, in the EPS and through fluid flow in channels within the biofilm, by means of chemicals exuded from biofilm cells; these systems are called “quorum sensing.” Up to forty-percent (40%) of individual microbial genes change expression as they assume different functions as a result of the transition from planktonic to biofilm state. There is also an exchange of genetic material between individual microbes of differing bacterial and fungal types within the biofilm. These pronounced physiological changes help biofilms to resist disinfection, to develop antibiotic resistance, to release deadly toxins and to break down materials. Exchanged genetic material confers additional resistance to an antibiotic, and this process leads to the rapid establishment of antibiotic resistance within the biofilm's contiguous population of pathogens. Accordingly, the control of microbial growth is an important issue in the broad field of science and public health as well as medicine.

Depending on the microorganism, the carrier, and ambient environmental conditions, microbial cells on a surface may proliferate, eventually resulting in formation of a biofilm. Because of decreased penetration into biofilms by diffusion and by transport within biofilm channels and also because of defenses presented by the EPS matrix of biofilms to various antibiotics and antimicrobials, animal tissue infected with a biofilm is more readily resistant to disinfection. Biofilms may allow microbial cells to survive under harsh conditions, and the embedded cells may be up to 1000 times less susceptible to antibiotics and biocides than were those cells in planktonic form. Bandages, debris and sloughed tissues have been discarded into the environment as partially treated waste allowing pathogens coming into contact with diluted and degraded compounds that have lost potency to withstand their effect and, in some situations, become resistant to such antibiotics.

Reliance-on antibiotics has resulted in microbial adaptation resulting in the creation of “superbugs” that are resistant to current clinical treatments. Less often recognized in the past, but more frequently the case in practice, microbial antibiotic resistance is due to microbes acting synergistically and in concert in a biofilm. This may be due to genetic adaptation resulting from Quorum Sensing or extracellular DNA within the EPS matrix, or it may be due to the development of cells that adopt a low metabolic state or dormancy (called Persister Cells) and become highly resistant to antibiotics. In this regard, it can be appreciated that there is a critical need for compounds that can be applied topically to destroy biofilms and not result in antibiotic resistance.

A great deal has been learned about biofilms, more of a general nature than of particularity though intense research is rapidly bringing new insights. Biofilms are a scientific area of great study in dentistry for plaque, in municipalities for water mains, and in medicine both for treatment of infection and for sterilizing everything from hospital rooms to surgical instruments. Current study about the minutiae of materials transport within the biofilm is ongoing and continuously revealing, but many aspects of biochemical interaction are incompletely understood. It is known that when the biofilm is moist or in liquid, channels within biofilm reliably carry microbial exudation through the EPS—exudation that allows for Quorum Sensing between microbes and also for liquids, nutrients and oxygen to penetrate the biofilm more rapidly than by diffusion. As biofilms grow in microbial population and diversity with exudation of a surrounding matrix of hydrated extracellular polymeric substances (EPS) they then undergo biologic organization through chemical signaling between microbes, facilitated by open and communicating channels within the biofilm layer through which liquids circulate to provide nutrition for microbes and egress for chemicals produced by the microbes. These channels, irregular and somewhat tortuous in form, have been measured at between 26 and 150 μM in diameter and when not excessively dehydrated, especially when in a humid or moist environment have been shown to allow a flow of liquids that permit tracking of experimentally introduced latex nano-particles or beads (identifiable by microscopy) within the channels. It is well known that molecular diffusion occurs in biofilms, but diffusion is far slower than transfer by flow through channels. A research article in 1996—ages ago in biofilm study—described antibiotic penetration of biofilms predicting that their marked loss of effectiveness was not adequately accounted for by diffusion or by differing gradients of pH or oxygen in layers of the biofilm.

It has been confirmed that antibiotics readily enter biofilms either by diffusion (more slowly) or by channels whereby the full depth may be penetrated within minutes to hours, but not all antibiotics or germicides are alike either in their abilities to traverse the channel nor in their efficacy at different strata levels, levels where oxygen and nutrition gradients differ and where the EPS content interferes with gene expression. Some antibiotics are slowed in their progress into the biofilm (e.g. erythromycin) as are some surficant/germicides (e.g. cetyltrimethyl ammonium bromide), while other antimicrobials like ciprofloxacin and benzalkonium chloride move rapidly into the biofilm, however the rate of channel flow or diffusion in penetrating a biofilm is not correlated with its killing or removal efficiency. Retardation of flow into the channels of biofilms seems to be related to differences among antibiotics in reactivity to substances in the EPS matrix. It seems that penetration differences are more a function of rate than inability to penetrate. Protection by EPS matrix, containing chemicals that alter gene expression and microbial adhesion (e.g. eDNA) and the presence of low-metabolism Persister Cells are the primary reasons for the lack of effectiveness of antibiotics, especially those antibiotics that are easily consumed by their killing reaction. And because of these factors, along with other variations in strata environment, antibiotic potency may be different in different strata of biofilms.

Within a biofilm, more so when it is older, thicker, there is spatial physiologic heterogeneity with the more metabolically active microbes being in the upper reaches and with persister cells residing in lower strata in a protective low metabolic state. Causation of persister cell metabolic slowing seems due to local conditions including nutrition gradient, oxygen gradient, pH gradient and gradients of waste products. Also, different antibiotics are more or less effective at different biofilm strata and against different declining levels of microbial metabolism (leading to full dormancy.) Mechanisms of microbial resistance tend to be specific for each microbe in relation to each antibiotic. Should an antibiotic fare better at the top level and fail at lower levels, the overall treatment would fail due to persister cells, and vice versa should the antibiotic work more poorly at upper levels. Some microbes within a biofilm adapt by presenting different gene expression as the biofilm is challenged with different antibiotics; this accounts for failure after a partially successful treatment when the second presentation of the same antibiotic (e.g. ciprofloxin or tobramycin) is met with altered resistance.

Quaternary ammonium compounds are widely used for clinical and industrial control of bacterial growth, and their effectiveness has not changed since their introduction in the 1930's. The antimicrobial action of quaternary ammonium compounds involves perturbation of cytoplasmic and outer membrane lipid bilayers through association of the positively charged quaternary nitrogen with the polar head groups of acidic phospholipids. Lethality occurs through generalized and progressive leakage of cytoplasmic materials. However, quaternary ammonium compounds have been of overall limited effectiveness in treating biofilms. Contact-Active Biocidal (“CAB”) quaternary ammonium compounds are pesticides that provide a non-toxic, non-leaching surface covering for inanimate objects. These products provide an invisible microbiostatic coating that kills single cell planktonic microbes which drift onto the treated surface. The CAB products are typically offered in liquid form and applied to surfaces such as walls or counter tops after disinfecting the surface or on clothing through a washing machine rinse cycle. Upon drying, the CAB bonds to the surface and establishes a killing field for new microbes that randomly come into contact with the coating. The effective life of the CAB product is relatively short. Moreover, once applied, it is difficult to determine at what time the biological activity becomes diminished and the CAB is no longer maintaining a disinfected surface. An undisclosed problem is a CAB not regularly cleaned can be expected to fill with dust and debris which creates an inviting Conditioning Layer and works counter to its claimed purpose. As a result, most CAB products used have not been a commercial success. Accordingly, there is a need for a different, novel and creative use for CAB compounds that addresses the concerns presented herein above; namely, providing an effective supplement for antibiotics in treating surface infections; inhibiting the transfer of microorganisms, body fluids and particulate matter; reducing use of toxic compounds that pollute the environment, and reduce the likelihood of treatment resistant strains which may spread and have deadly consequences.

### DISCLOSURE OF EMBODIMENTS OF THE INVENTION

The present disclosure relates generally to new and novel use of antimicrobial compounds (collectively called “Silane Compound”) in particular, to organosilane quaternary ammonium compounds for killing planktonic microbes, for disruption of a biofilm, for interfering with the communication between biofilm cells and purturbation of the homeostasis of its matrix for treatment of infections of eyes, ear canals, skin and subcutaneous tissues in animals and humans, controlling biofilm colonization of wounds and animal tissue surfaces, and treatment of other microbial pathogens found in infections involving same.

Disclosed is an antimicrobial method of action comprising an organosilane; a carrier; and a delivery system. The Silane Compound method of action has two components, ionic and mechanical. First positive charge on the N+(nitrogen atom) of the molecule causes attraction to the microbe, ionically weakening the cell wall, causing increased permeability, and the long carbon chains abut, entangle and puncture the weakened cell wall. This dual method of action assures the destruction of the cell. This molecule has the additional benefit of covalently binding the compound to the surface of the area treated and remaining effective until the tissue is sloughed or replaced. This allows for potential destruction of any persister cells that survive the initial attack. Finally, the compound is non-toxic and will not harm the environment or promote resistance to Silane Compound or other antibiotics.

The molecular size of 3-(trihydroxysilyl) propyldimethyloctadecyl ammonium chloride in Silane Compound is about 28-30 nM in height and 10 nM wide, small enough to traverse the biofilm channels. The channels, being necessary for biofilm functions (that include Quorum Sensing) permit Silane Compound to flow along with nutrients within the channels. The positive charge through the presence of the N+(nitrogen) atom of the 3-(trihydroxysilyl) propyldimethyloctadecyl ammonium chloride (hereafter called Trihydroxysilyl Quat) molecule is important in aiding flow and in providing ionic attraction to the negative microbial cell walls.

Oxygen gradients are lowest in the deepest layers of the biofilm, a condition that is thought to be among the most critical in promoting the formation of persister cells. Channels allow flow of liquids to reach the lowest layers of the biofilm and would permit molecules the size of Silane Compound to reach persister cells in the base layer. Within the channels, the positive charge of the nitrogen atom attracts the commonly negatively charged cell wall of the microbe and by affecting the phospholipids and proteins of the cell wall, make it more permeable and the silane carbon chains can contact, penetrate and entangle microbial cell walls. The killing action of Silane Compound is the same for active and persister cells as long as the target cell has a negative charge on its cell membrane. Thus, by first being active in the upper layer of the biofilm, Silane Compound with treatment protocol of several days, can reduce the population of microbes and the thickness of the biofilm. This makes the lowest level more accessible and is likely to create improved nutritional and oxygen gradients promoting an increase in the metabolic activity of the microbes located at the base. Silane Compound activity, unlike that of many antibiotics that are consumed by their killing action, would not necessarily be deactivated after killing a single microbe, and with collapse of a microbial cell, it may remain in flow or in the EPS of the film to kill cells in lower layers.

Improvement by using the Trihydroxsilyl Quaternary Ammonium Compound seems due to the combination of the known ionic effect of cell wall damage plus the long carbon chains that physically contact, entangle and pierce the weakened cell wall. Silane Compound's killing method is not affected by EPS matrix reactivity as demonstrated by our preclinical evidence.

Silane Compound is intended to cure serious skin infections and to prevent complications leading to cellulitis, abscess formation or septicemia. The skin is the largest organ of the body. The adult skin weighs about 8 pounds with an area of about 22 square feet. It acts to sense stimuli, to regulate temperature and to protect the body. But, it can also become infected. Each area of the skin is different in hair bearing, in color, in oil glands, in perspiration, in blood flow and in healing and resistance to infection. The scalp, peri-rectal and facial areas tend to heal well and resist infection, while lower extremities tend to heal more slowly. The skin of the ear canal in dogs, for example, can become a reservoir of infection. Biofilm skin infections are many times more resistant to treatment and can support a mixed population of microbes that lead to cellulitis, abscess formation and life threatening septicemia. Because of skin's quantitative and qualitative importance, effective treatments of infections of the skin are crucial not only for the well-being of a human and animal but for its very survival. Silane Compound provides an effective substitute for existing medications, brings long term relief and promises to improve all aspects of clinical treatment for skin infections. Since Silane Compound is not an antibiotic it lacks the disadvantages of antibiotics in promoting microbial resistance.

In some embodiments, the delivery system is a microcapsule enclosing the 3-(trihydroxysilyl) propyldimethyloctadecyl ammonium chloride molecule therein or a microparticle coated with the organosilane. In some embodiments, the concentration of the organosilane is less than 0.10 percent by weight. In some embodiments, the concentration of the organosilane is between 0.10 percent and 1.00 percent by weight. In some embodiments, the concentration of the organosilane is greater than 1.00 percent by weight. In some embodiments, the concentration of the organosilane is greater than 10.0 percent by weight. In some embodiments, the carrier is a compound selected from the group of carrier compounds consisting of: water, an alcohol, polyethylene glycol or a petroleum-based ointment.

In some embodiments, Silane Compound, further comprises an enzyme. In some embodiments, the enzyme is a proteolytic hydrolase enzyme. In some embodiments, the enzyme is an enzyme acting upon a substrate comprising N-acyl homoserine lactone.

In some embodiments, Silane Compound further comprises a detergent. In some embodiments, the detergent is a quaternary ammonium compound. In some embodiments, Silane Compound further comprises an anti-inflammatory. In some embodiments, the anti-inflammatory comprises a steroid molecule. In some embodiments, the anti-inflammatory is a compound selected from the group of anti-inflammatory compounds consisting of: hydrocortisone, triamcinolone diacetate, beta methasone valerate, beta methasone diproprionate, resorcinol, and methyl resorcinol. In some embodiments, Silane Compound further comprises an antiseptic. In some embodiments, the antiseptic is a compound selected from the group of antiseptic compounds consisting of-hexylresorcinol, methyl resorcinol, and ethyl alcohol. In some embodiments, Silane Compound further comprises a topical anesthetic. In some embodiments, the topical anesthetic is a compound selected from the group of topical anesthetic compounds consisting of: lidocaine hydrochloride, and benzocaine hydrochloride. In some embodiments, Silane Compound further comprises a penetrant to aid in treating keratin. In some embodiments the penetrant is DMSO, dimethylsulfoxide.

In some embodiments Silane Compound contains a buffer selected from the group of buffer compounds consisting of: a citrate, a sulfonate, a carbonate, and a phosphate.

In some embodiments, the antimicrobial product further is accompanied by a fragrance, wherein under a condition wherein the microcapsule is broken, the antimicrobial product is adhered onto an article and the fragrance becomes active; and wherein a time of useful antimicrobial activity of the activated antimicrobial product corresponds to a time wherein a scent of the active fragrance is present. In some embodiments, the organosilane is a 3-(trimethoxysilyl) quaternary ammonium compound or a 3-(trihydroxysilyl) quaternary ammonium compound.

Disclosed is a method of treating an infection, the method comprising steps of applying an aqueous antimicrobial product comprising an organosilane to a human or animal tissue substrate; killing a microbial cells and biofilms; and establishing a method of use that is effective to treat reactivation of any remaining persister cells. Treatment of skin infections requires repeat applications. As the tissue infection is reduced, as access becomes more ready with decreased swelling, and as the biofilm is destroyed and more tissue becomes exposed, more of the silane may become bonded to the tissue substrate. The residual Trihydroxysilyl Quaternary Ammonium Compound will continue to kill revived planktonic microbes until the tissue sloughs or the skin is shed. By way of clinical observation (see below) a highly resistant dog, “Conner,” has been infection free using drops only in each ear every few weeks.

Disclosed is a method of providing a non-toxic antimicrobial treatment to a dressing, gauze, spunlace, nonwoven, melt blown or other materials, the method comprising the steps of applying a delivery system for a product containing an organosilane; activating the delivery system at the time of use; and adhering the organosilane.

Disclosed is a method of providing a non-toxic antimicrobial treatment to dressings for site infections and filtering materials, the method comprising the steps of embedding a surface during manufacture of the surface of the item with a microcapsule enclosing an antimicrobial product comprising an organosilane and releasing the antimicrobial product from the capsule.

In some embodiments, the method further comprises a step of placing treated material in proximity to an area, a container, of microbial biofilm presence.

The foregoing and other features and advantages of the present invention will be apparent from the following more detailed description of the particular embodiments of the invention, as illustrated in the accompanying drawings.

### BRIEF DESCRIPTION OF THE DRAWINGS

Some of the embodiments will be described in detail, with reference to the following figures, wherein like designations denote like members:

FIG. 1 is a schematic diagram showing a general chemical structure of a Trihydroxysilyl organosilane molecule;

FIG. 2 is a schematic diagram showing a general chemical structure of a Trimethoxysilyl organosilane molecule;

FIG. 3 is a schematic representation of a delivery system comprising a microcapsule for an antimicrobial product;

FIG. 4 is a schematic representation of a delivery system comprising microcapsules enclosing an antimicrobial product, an aging indicator, and a fragrance;

FIG. 5a is schematic representation of a biofilm showing a general configuration of microbial cells and EPS, extracellular polymeric substance, with fluid-filled channels containing molecules of 3-(trihydroxysilyl) propyldimethyloctadecyl ammonium chloride;

FIG. 5b is a zoomed in schematic representation of a biofilm showing a general configuration of microbial cells and EPS, extracellular polymeric substance, with fluid-filled channels containing molecules of 3-(trihydroxysilyl) propyldimethyloctadecyl ammonium chloride:

FIG. 5c is an even greater zoomed in schematic representations of a biofilm showing a general configuration of microbial cells and EPS, extracellular polymeric substance, with fluid-filled channels containing molecules of 3-(trihydroxysilyl) propyldimethyloctadecyl ammonium chloride;

FIG. 6 is a diagram of a method 300 of treating infection and/or infectious disease and/or providing short term disinfectant properties to a dressing or filter; and

FIG. 7 is a diagram of a method 400 of forming an article with an antimicrobial product delivery system embedded therein.

### DETAILED DESCRIPTION OF EMBODIMENTS OF THE INVENTION

A detailed description of the hereinafter described embodiments of the disclosed method are presented by way of example and not meant to be limiting with reference to the Figures listed above. Although certain embodiments are shown and described in detail, it should be understood that various changes and modifications may be made without departing from the scope of the appended claims. The scope of the present disclosure will in no way be limited to the number of constituting components, the materials thereof, the shapes thereof, the relative arrangement thereof, etc., and are disclosed simply as an example of embodiments of the present disclosure.

As a preface to the detailed description, it should be noted that, as used in this specification and the appended claims, the singular forms “a”, “an” and “the” include plural referents, unless the context clearly dictates otherwise. Some general definitions are provided for the terms used herein. “Biofilm” is any group of microorganisms in which cells stick to each other on a living or non-living substrate. These adherent cells are commonly embedded within a self-produced matrix of extracellular polymeric substance (EPS). Microbes in a biofilm state make collective decisions by communicating with chemical signals called “quorum sensing.” The microbial cells growing in a biofilm are physiologically distinct from planktonic cells of the same organism, which, by contrast, are single-cells that may float or swim in a liquid medium. “Organosilane” means a compound of the family of compounds comprising the elements of silicon, oxygen, and carbon with a C—Si covalent bond and a nitrogen atom in a quaternary ammonium configuration. “Organosilane” also includes any quaternary ammonium salt of an organosilane. “Microbial cell” and “microbe” are used interchangeably and are understood to mean any single-celled micro-organism, bacteria, yeast or fungus. “Microcapsule” refers to a subset of the broader category of “microparticles,” wherein the microcapsule is a microparticle having a core comprising one material or compound surrounded by a distinctly different second material or compound. As the generally accepted size range for microparticles, a microcapsule has a size within the broad range of 1 micron to 1000 microns (1 millimeter). Therefore, the size range of a microcapsule, for the purposes of this application, is between that of a large nanoparticle to an object visible to the eye without magnification. Microparticle may also refer to a solid compound comprising the particle that is, itself, coated with the organosilane for purposes of becoming imbedded in a conditioning layer or more mature biofilm. “Animal Tissue” means the skin, epidermal linings, dermal subcutaneous tissues, granulation tissues, corneas, and outer membranes of eyes and ear canals of vertebrates, on which microorganism(s) are attached.

Disclosed is a method of use of an antimicrobial product 100 (Silane Compound). Product 100 is an organosilane 102 in combination with other compounds in a mixture chosen according to the intended application of product 100.

The antimicrobial action of product 100 is provided by the organosilane. An organosilane is a molecule comprising a silicone atom covalently bonded to carbon. Organosilanes in general may be amphiphilic, having both water-soluble and lipid soluble components. Organosilane 102 comprises a hydrophilic “cap” comprising a silicon-tri-methoxy or silicon-tri-hydroxy “head,” and a hydrophobic “tail” comprising an eighteen or twenty-atom linear carbon chain. The head and tail are joined at a nitrogen atom bonded with two additional methyl groups to create a (cationic) quaternary ammonium group. The Trihydroxy head groups facilitate enzymatically or chemically binding the organosilane to a human or animal tissue substrate 140. The positively-charged hydrophilic quaternary ammonium group allows for ionic attraction to the negatively-charged microbial cell common to most bacteria and fungi. Once being attracted to the microbes negative cell-wall charge, the linear hydrophobic hydrocarbon tail of the organosilane engages, entangles or penetrates the already damaged phospholipid cell membrane, disrupting the membrane, causing lysis with death of the cell. This microbial killing mechanism is advantageous for several reasons. Organosilane 102 is not altered or consumed by its interaction with the targeted microbe. The organosilane is non-toxic and will not adversely impact the environment.

In various embodiments of the invention, other adjuvant compounds are added to product 100. For some embodiments wherein product 100 is used in healthcare and veterinary medicine application, an anti-inflammatory 103 is added to reduce inflammation and itching. In some embodiments, a topical anesthetic is added to reduce pain and itching from inflammation to prevent scratching. For some embodiments wherein product 100 is used to kill microorganisms in a biofilm product 100 further comprises a cellulase enzyme. In some embodiments, product 100 further comprises other enzymes or compounds to aid in breaking down the EPS matrix of the biofilm. In some embodiments, product 100 comprises an agent to modify viscosity. In some embodiments, product 100 comprises an agent to promote trans-epithelial delivery of un-bound product 100 through skin and keratin. For some embodiments a keratolytic is added to aid in penetration. For some embodiments, dimethylsulfoxide is added to help penetrate the keratin of nails.

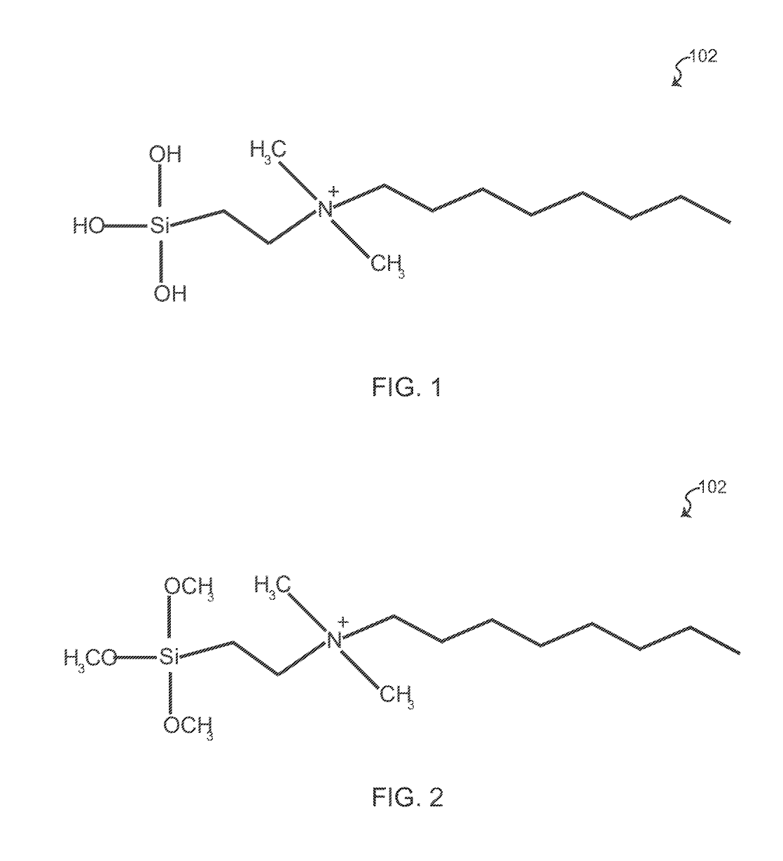

Referring to the drawings, FIG. 1 and FIG. 2 each depict an organosilane 102. Neither of these organosilnes have been previously approved for use to cure infections in humans or animals. The Trimethylsilyl Quaternary Ammonium compound (Trimethoxysilyl Quat) is illustrated only as a precursor to the Trihydroxysilyl form, when hydrolyzed by addition to water. The Trimethoxysilyl Quat is unstable in water and is often transported in 40 to 50% menthol. The traditional method of use of the Trimethoxysilyl Quat is to add a dilute solution in methanol to water. The EPA label required for Trimethoxysilyl Quat compounds states: “Danger. Corrosive. Causes irreversible eye damage and skin burns. Methanol may cause blindness. May be fatal if inhaled. May be harmful if swallowed or absorbed through the skin.”

Trimethoxysilyl Quat in water undergoes hydrolysis and then through condensation, covalently bonds to a surface. The result of hydrolysis of 3-(trimethoxysilyl) propyldimethyloctadecyl ammonium chloride is {RSi(OMe)3+3H2O-3 MeOH+RSi(OH)3} and the formation of 3-(trihydroxysilyl) propyldimethyloctadecyl ammonium chloride, a molecule that is stable with no other additives in water. The compound must then be used within a short period of time, such as a few hours to at most 12 hours, to treat a surface or fabric to produce a permanent surface coating. The presence of methanol makes this compound a public health hazard as it is unsuitable for use as a drug.

When used as a pesticide on inanimate objects, bonding to a cleaned and often sanitized surface and drying is key to the effectiveness of the Trimethoxysilyl Quat. During the drying process the Trihydroxysilyl molecules condense on the surface and strongly bond covalently. “By permanently altering the surface it is applied to, the silane monomer cross links to itself, polymerizes and imparts a durable protective barrier against microbial attack.” It is important to note that this defense is against planktonic microbes and that the delivery system, involves drying and bonding to prevent but not to treat biofilms. It is also the case that the surface protection created by these bonded molecules depends on regular cleaning since the barrier is only 20 to 40 nM in depth and is made ineffective when covered over by micro-detritus and debris. Bacteria range in size from 120 nM to 1000 nM and so coverage of the barrier by detritus from killed microbes is a problem. In fact, detritus and debris may become a conditioning layer if not regularly cleaned.

It is important to emphasize that Silane Compound, the animal tissue product, does not use the Trimethyl molecule but instead the Trihydroxy molecule because of its stability in water thus allowing its use without the toxicity of methanol. The microbial killing ability of Claim 29 and Compound 100 is due to molecule 102, 3-(trihydroxysilyl) propyldimethyloctadecyl ammonium chloride and not the carrier or any added adjuvants. The novelty of Silane Compound is not that of a newly discovered molecule but in its target (established infections of the skin, including both planktonic microbes and biofilms) and also method of delivery using liquid form directly applied to the infected tissue, without drying. The molecule (Trihydroxysilyl Quat) has not previously been approved by the United States Federal Drug Administration as a drug (Environmental Protection Agency classification is as a pesticide) for treatment of an established infection, and so there are no comparable animal uses for the Trimethoxy/Trihydroxy-silyl group.

These non-limiting examples show the fundamental structure of two organosilanes 102 with antimicrobial activity. Common to organosilanes 102 are a silyl “head,” a quaternary ammonium group, and an aliphatic hydrocarbon “tail.” Embodiments of product 100 comprise organosilane 102 in the form of the Trihydroxysilyl Quat, and in some embodiments, additional structural and functional components, adjuvants that complement one another to add functionality and performance to product 100, the structure and function of which will be described in greater detail herein.

In the example embodiment shown in FIG. 1, organosilane 102 is a 3-hydroxysilyl organosilane. The silyl “head” of the molecule is shown to the left of the figure, comprising three trihydroxyl groups which, in some embodiments, are reacted to covalently bond with a biological or non-biological substrate. The quaternary ammonium group is also shown, connecting the silyl “head” with the aliphatic hydrocarbon “tail.” In the example embodiment shown in FIG. 2, organosilane 102 is a 3-methoxysilyl organosilane. In Compound 100, organosilane 102 is a 3-(trihydroxysilyl) propyl dimethyl octadecyl ammonium molecule. A difference in ionic charge, the positive being the organosilane Nitrogen atom and the negative being the cell walls of most microbes, causes attraction. perturbation of the cell wall with damage and weakening of the cytoplasmic membrane causing breakdown, increased permeability and leaking, and further it is believed that continued destruction of microbial cells 135 occurs by engagement with the carbon chain hydrophobic “tail” of organosilane 102 causing physical disruption and death.

In some embodiments involving treatment with product 100 of an infected animal tissue substrate wherein inflammation and edema are present, an anti-inflammatory compound is a useful therapeutic adjunct to reduce swelling, gain additional access (in the case of an ear canal for example) and to reduce itching and scratching. Microbial infection normally creates an inflammatory response. Inflammation creates local swelling, increases pain and/or itching, and, if marked may interfere with healing. This is particularly important in veterinary application wherein inflammation compels the animal to scratch at the infected substrate, aggravating inflammation and leading to additional irritation and more scratching. Skin and ear canal infections are so common as to be a leading cause of visits to a veterinarian, too often after weeks of home treatment have failed. Incidence numbers are so large as to be a serious problem if only on economic grounds. Difficult cases however are not rare, and for companion animals with allergies, eczema, or diabetes the disease is serious in degrading life-style through pain, itching and wasting of general health. In particular, with repeated occurrence—a common problem for several breeds—this morbidity has substantial negative effect on day to day functioning, including itching, pain stenosis of ear canals and hearing loss. In veterinary medicine, otitis externa is a serious problem particularly with biofilm formation and in dogs with stenosis of ear canals. Also, in the case of lacerations, particularly those that may expose tendons, the resulting tendinitis can lead to loss of a limb or loss of the animal's life.

Therefore, treatment with a topical or systemic anti-inflammatory compound is often useful. And so, in some embodiments, product 100 further comprises an anti-inflammatory molecule. Some non-limiting examples of such anti-inflammatory compounds include steroids, such as triamcinolone diacetate, hydrocortisone, beta methasone valerate, and beta methasone diproprionate. In some embodiments, product 100 further comprises a topical anesthetic to treat the pain and itching associated with the inflammatory response, some non-limiting examples including lidocaine hydrochloride, benzocaine hydrochloride, hexylresorcinol and methyl resorcinol.

### Experimental Examples

Preliminary clinical evidence for Silane Compound has demonstrated substantial improvement over existing therapies with regard to effectiveness, cost, frequency of use, reduction of chronicity due to biofilms and persister cells, and thus serves an unmet medical need for companion animals. These benefits will be discussed below, but it is convenient to list Silane Compound's advantages over existing therapies in that:
It does not promote persister cell resistance because it kills microbes and effectively disables, destroys biofilms.
Its pattern of use, twice daily or once daily, promotes compliance
Its low toxicity address unmet needs that allow for environmental use.
It will treat conditions in animals that are intolerant to or allergic to previously tried antibiotics; there is not cross reactivity with antibiotics.
It will bond to tissue and linger in the treated area acting as treatment should persister cells reactivate.
It provides efficacy against treating yeast, fungus and bacterial infections with a single agent.
It offers cost savings for several of the reasons above: the cost of this single agent is lower than the acquisition cost of either antibiotic or antifungal agents and greatly less costly than providing two antimicrobials in a single compound.

In one experimental example, a formulation of product 100 comprising organosilane 102 was successfully used to treat a severe long-standing case of a mixed bacterial/fungal infectious otitis externa and infectious dermatitis in a Shih Tzu dog. “Conner,” a neutered male Shih Tzu, aged 11 years 7 months, presented for re-evaluation of bacterial and monilial dermatitis (Malassezia sp.). Conner had a past history of keratoconjunctivitis sicca, generalized demodicosis, and allergies. The evaluation and subsequent treatment with product 100 was begun two months after failed conventional therapy with oral antibiotics (amoxicillin/clavulanate (Clavamox® 13.6 mg/kg) orally b.i.d. for four weeks) and an oral antifungal (fluconazole, 5.4 mg/kg once daily for two weeks; then once every-other-day for two additional weeks.) The earlier failed therapy also consisted of bathing the animal 2-3 times a week using an anti-seborrheic shampoo (KeratoLux®) and an antimicrobial shampoo (Duoxo Chlorhexidine® shampoo) followed by an oatmeal-based cream rinse (Episooth®). The Conner's chest, neck, paws, and face were cleaned and treated twice daily with antimicrobial wipes (Douxo Chlorhexiding Pads®) and an antimicrobial lotion (ResiKetoChlor®). The ears were cleaned once daily with a tris-EDTA/ketoconazole solution (TrizUltra plus Keto®) and treated twice daily with an amikacin otic preparation.

Dermatologic examination revealed extremely abundant purulent exudate in both ears with stenotic canals and erythema, lichenification, and edema on both medial pinnae. The animal had generalized mixed hypotrichosis/alopecia, erythema, hyperpigmentation, and lichenification with crusting over dorsal trunk, legs, paws, and ventrum. Hair on the face and neck was severely matted. The skin underlying the mats was crusted, with moist dermatitis and brown purulent exudate. Lymph node enlargement was palpated in the mandibular, prescapular, and popliteal node groups.

Cytological examination of skin scrapings revealed abundant bacterial dermatitis with cocci and diplococci. A generalized severe Malassezia (yeast) dermatitis was concentrated mostly on ventrum and paws. Otic examination revealed bilateral severe bacterial otitis externa with cytological examination revealing abundant mixed population of rods and cocci, along with occasional Malassezia. Demodex canis was seen in all life stages. Fine-needle aspirates of peripheral lymph nodes (left prescapular and left popliteal) were consistent with reactive lymphadenopathy.

The otitis externa was treated initially by cleaning the ears with a salicylic acid based ear cleaner (Otoclean®) and instillation of 0.5 ml of product 100 comprising organosilane 102 (3-(trihydroxsily) propyldimethyloctadecyl ammonium chloride) in each ear. Mats were removed and the dog was bathed and groomed over two days using (Splash Plus® shampoo), followed by antimicrobial shampoo (Douxo Chlorhexidine® shampoo), followed by essential fatty acid cream rinse (Hylyt® cream rinse). After bathing, product 100 comprising organosilane 3-(trihydroxsily) propyldimethyloctadecyl ammonium chloride was applied to facial folds and over body using moistened gauze sponge pads.

“Conner” returned 7 days later for a brief recheck. Otic cytology revealed complete resolution of the bilateral bacterial otitis externa and significant improvement of the bacterial dermatitis at all sites. Only a mild amount of ceruminous exudate was observed in each ear. Lichenification and moist dermatitis had decreased substantially. No purulent exudate was observed at any site. Topical therapy was repeated (bathing with Splash Plus® shampoo, Douxo Chlorhexidine® shampoo, and Hylyt® cream rinse) followed by application of product 100 comprising organosilane 102 (3-(trihydroxsily) propyldimethyloctadecyl ammonium chloride) using moistened gauze pads. Otic therapy was repeated with a cleaning using salicylic acid based car cleaner (Otoclean®) and treated by instilling product 100 comprising organosilane 102 (3-(trihydroxsily) propyldimethyloctadecyl ammonium chloride) in each ear. Weekly rechecks over the course of one month showed continued improvement and no recurrence of bacterial or Malassezia infections.

In a second experimental example, “Lillie,” a fawn Puggle born April 2009, was seen for an acute episode of otitis in her left ear in November 2014 after no previous history of any trauma, bathing, swimming, or medical issues. A purulent discharge was exuding from the ear canal and inflammation was apparent in the aural pinna on both the anterior and posterior aspect. A culture swab was collected for Clinpath® ID and sensitivity and product 100 comprising organosilane 102 (3-(trihydroxsily) propyldimethyloctadecyl ammonium chloride) solution with steroid was directed to be flushed daily into the car canal after the ears were cleaned utilizing a Q-tip soaked in a general cleansing solution.

Results of the culture and sensitivity showed a mixed infection of Pasteurella Multocida 1+, coagulase positive Staph species 3+, and Malassezia yeast 1+. Flush medication was continued for two weeks and the car was reexamined. The external auditory canals were wide open and showed no purulent debris or inflammation indicative of any previous infection. General cleaning on a regular basis was recommended with Epi-Otic®. [0057] After 1 month, Lillie returned with a second bout of otitis occurring post grooming. It was learned that the groomer had been applying a medication in the ears for “ear mites” and the infection started within 48 hours post application. The owner was again instructed to flush the ears with product 100 comprising organosilane 102 (3-(trihydroxsily) propyldimethyloctadecyl ammonium chloride) solution including a steroid and have them rechecked in 1 week. Within 1 week the owner called back to report that the ears had cleared up and that she would not need the recheck appointment.

Lillie has since been seen for underlying allergies and has been itching at her ears causing sores and lesions on the external pinna. Otic cytology revealed no etiologic agent, just inflammatory and epithelial cells. Although the generalized erythema has become more prevalent on Lillie's body, her ears have been able to remain infection free to this point with weekly routine cleansing and flushing with the product 100 solution.

In a third clinical example, a clinic owned, 5-year-old yellow lab named Seger who was seen in a veterinary hospital in December of 2014 and was diagnosed with chronic bilateral otitis. He had been treated in the past at other veterinarians' offices with regular ear cleansers, “zymox”, epiotic, and Triz EDTA. Multiple antibiotics and formulations that contain topical antibiotics were utilized along with cleaning, but with reoccurrence happening within several weeks after cessation of meds. The most commonly used antibiotic had been gentamicin, and the anti-fungal nystatin or clotrimazole in preparations such as Otomax, Mometamax, or Panalog. These compounds also contained small amounts of steroids for inflammation. BNT was also used as a weekly packing. This contained enrofloxacin, nystatin, and triamcinolone.

Cultures initially produced results that included mixed infections of cocci/rod bacteria as well as 2-3+ yeast. In March of 2015, Seger was cultured again and was diagnosed with a 4+ Pseudomonas Aeruginosa infection in both ears along with a 1+ yeast isolate. At this time Seger's attendant was instructed to flush ears daily with Silane Compound and recheck in two weeks.

Upon recheck, the purulent discharge was nearly gone and aural skin ulcerations were clearing up significantly.

Seger was seen again two weeks later (4 weeks after initially starting treatment) and all of his otitis had cleared up and no sores or discharge were noted in either canal. Owners were instructed to use as a routine cleanser/treatment weekly thereafter.

As the caregivers ran out of their supply of the medication, Seger was seen again in October for otitis. He had no complications during his entire treatment with Silane Compound.

Some antibiotics and enzymes function optimally within a relatively narrow pH range. Accordingly, some embodiments of product 100 add a buffer to the treating compound at concentration levels sufficient to maintain the pH range required for optimal activity of the components of Silane Compound. The particular buffer is selected based upon the local conditions present on the biological animal tissue substrate. Buffers to maintain ambient pH within a desired range include, but are not limited to, citrates, sulfonates, carbonates, and phosphates. The preferred buffering compound and concentration of same useful for maintaining a desired pH range are dependent on ambient micro-environmental conditions at the treated area and known to those skilled in the art.

Microbial cells 135 may be bacteria, archaebacteria, protists, or fungi, and in particular may be cells that have survived destruction of the biofilm, the so-called persister cells. Persister cells become dormant as they live in lower strata of the biofilm that have gradients of lower oxygen and nutrition than strata above; this gradually causes the microbe to reduce metabolic activity leading eventually to dormancy and extreme resistance to being killed by antibiotics. As long as there is a negative cell wall charge on the microbial cell wall, organosilane 102 can function to kill the microbe. Should the cell wall charge be reduced or absent through dormancy, the cell will reactivate when its environment is propicious, usually thought to be when more oxygen and nutrients are present. The method of treatment herein described kills reactivated persister cells and thus reduce the chronicity of infections.

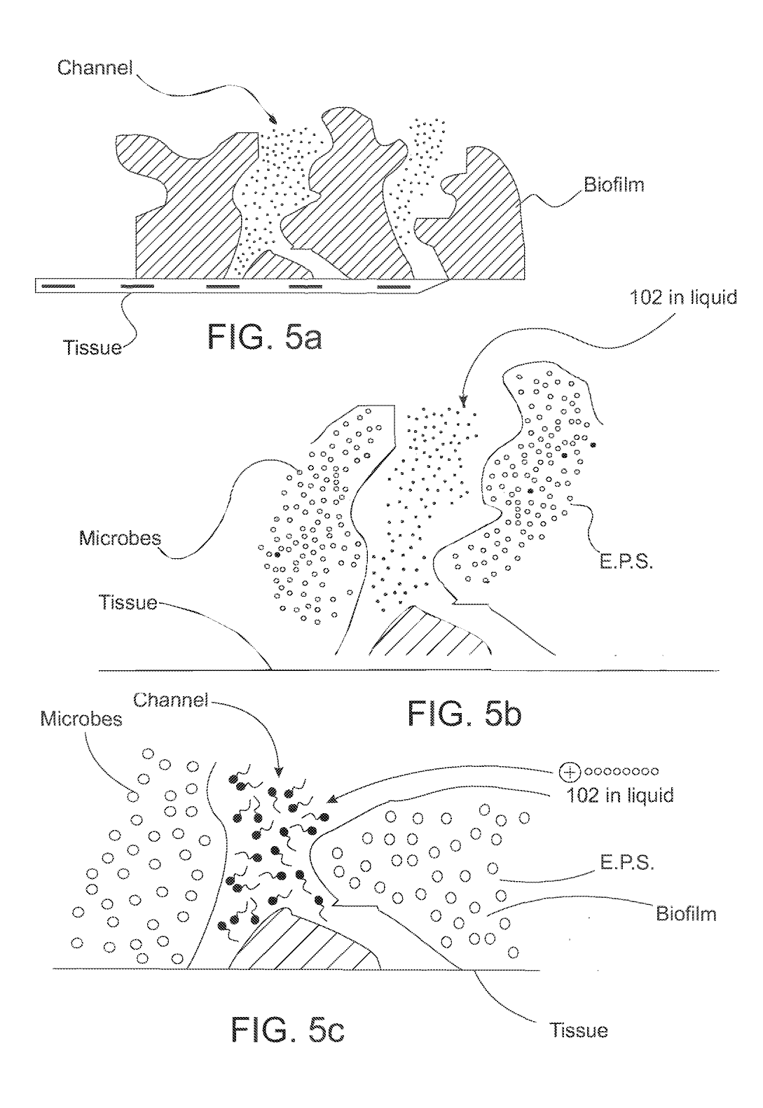

Referring to FIGS. 5a-5c, a microbe generally carries a negative net charge on the cellular wall due to constituent membrane proteins. For example, the cell walls of Gram-positive bacteria contain negatively-charged teichoic acids. The cell membranes of Gram-negative and Gram-positive bacteria (and other microbes) comprise negatively charged phospholipid molecules. Microbes, therefore, are ionically attracted to cationic compounds, such as the quaternary ammonium group-containing organosilane 102 that may attach to an animal tissue substrate or are free-floating in liquid biofilm channels, and may join to the cell wall and cytoplasmic membrane. If the compound, such as organosilane 102, is amphiphilic, the hydrophilic portion of the molecule may traverse both the bacterial cell wall and cytoplasmic membrane, causing cellular lysis and death of microbial cell 135. As a result, the attraction and joining of product 100 comprising organosilane 102 to animal tissue substrate 140 results in substrate 140 becoming configured to kill reactivated persister cells 135 on contact. Because this substrate killing does not disrupt and consume product 100, frequency of application of medication is reduced and microbial killing is accomplished without releasing a biocide to the environment.

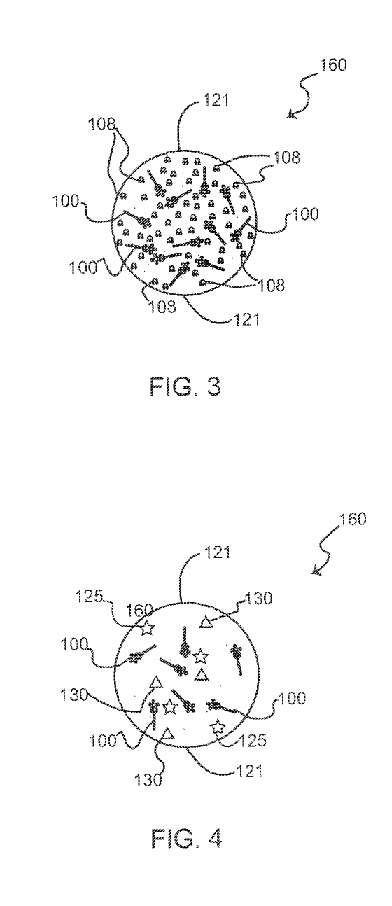

Product 100 additionally comprises a carrier 108, schematically shown in FIG. 3. Carrier 108, in some embodiments, is a compound that holds the various sub-components of product 100 in suspension or solution. The specific compound used is chosen based upon the characteristics necessary for the end-use application of product 100. For example, if product 100 is to be used on a substrate, such as skin lining the external auditory canal of a dog, carrier 108 may be an emollient, wax, alcohol, non-ionic surfactant, or other suitable compound. The carrier is, in some embodiments, a gel, lotion, ointment, aqueous liquid solution suspension, according to the intended end-use of product 100.

The concentration of organosilane 102 by weight of product 100 is also selected according to the desired end-use of product 100. In situations where high antimicrobial activity is needed for treating a well-established biofilm, concentrations of organosilane provide a higher density of organosilane molecules. surrounding the biofilm and in the communicating channels of the biofilm. In effect, the “cloud” of aliphatic hydrocarbon molecular “tails” is more dense. Additionally, higher organosilane concentrations create a higher cationic charge density, resulting in both stronger electrostatic microbial attractive forces and detergent effects on the microbial phospholipid cell membrane. Concentrations of organosilane 102 in product 100 of up to and over 5% by weight may be used, however, when used in concentrations of over about 3%, polymerization of organosilane 102 within product 100 increases through intermolecular cross-linking via —S—O—S— covalent bonds. In applications to substrates, such as a cutaneous epithelium or an open wound treated using product 100, product shedding through epithelial turnover may requires re-application of product 100, in some applications. The risk of bacterial and other microbial resistance to an antimicrobial compound, regardless of the mechanism of action of the compound, theoretically increases with increasing environmental encounters between bacteria and other microbes, and the antimicrobial compound. It is prudent, therefore, to strive to minimize the amount of any composition with antimicrobial activity within the general environment. Accordingly, in the aforementioned and other situations wherein regular re-application of product 100 is necessary, lower concentrations of organosilane 102, in product 100, are useful by lowering the overall amount of organosilane 102 ultimately discharged into the environment. Notwithstanding the theory, it is believed that the risk to the environment and/or causing biofilm mutations by use of these formulations is minimal.

Advantages of product 100 according to the several embodiments described herein, include the aforementioned non-leaching properties of product 100; decreased risk of microbial resistance giving the unique mechanical disruption of cell walls and cell membranes common to all microbes; the relative non-toxicity of organosilanes when used to treat an animal tissue substrate infection; and the stability of product 100 when bonded to animal tissue substrates, decreasing the need for frequently repeated application and subsequent dispersal of organosilanes and other constituent compounds of product 100 into the environment.

In some embodiments, product 100 and/or delivery system 160 is applied to an existing biofilm as a liquid using a dropper. In some embodiments, product 100 is applied as an aerosol, other spray, or wiped as a gel or ointment onto an animal tissue substrate. One non-limiting example is wherein product 100 is applied in liquid form to a well-established biofilm within the chronically-infected external auditory canal of a dog, as in the case study described herein above.

FIG. 4 shows a microcapsule 121 encasing product 100. Microcapsule 121 is one example of delivery system for product 100. Microcapsule 121, in some embodiments, comprises a material enveloping and containing product 100. Non-limiting examples of compounds used to form microcapsule 121 include polyvinyl alcohol, cellulose acetate phthalate, gelatin, ethyl cellulose, glyceryl monostearate, bees' wax, stearyl alcohol, and styrene maleic anhydride. Many other compositions of microcapsule 121 are possible, and the exact composition, construction, and manufacture of microcapsule 121 is chosen from the broad range of compositions and manufacturing techniques for microcapsules generally, and which are readily available and known to those skilled in the art. An example of such delivery system product 100 is encapsulated within microcapsule 121 and thereafter released when microcapsule 121 is broken. Breakage of microcapsule 121 is effected at a chosen time and in a manner specific to the particular use of product 100. For example, microcapsule 121 may be broken by scratching, as when a dog scratches the ostium of its external auditory canal in response to itching arising from inflammation. In this manner, product 100 is configured to be released to treat the symptom-producing area. Another example is incorporation of a microcapsule is incorporation in a face mask, filter or gauze. Because product 100 becomes active upon breaking of microcapsule 121, the effective useful life of product 100 begins within a time frame controlled by the end user based on when antimidrobial is most needed. When antimicrobial activity decreases, the thin-layer of product 100 and any associated, entangled cellular debris is removed from the surface prior to re-application of fresh product 100 (not on cloth.) In addition to protcolytic keratinases, some embodiments of product 100 comprise other enzymes. For example, N-acyl homoserine lactone is a bacterially-produced amino sugar acting as a hormone involved in quorum sensing, wherein a population of bacteria limits its growth density and other population-based characteristics, such as gene regulation of enzyme systems and the expression of flagella versus pili.

Some embodiments of delivery system 160 for product 100 require application of a direct force to the treated surface 140 by the user. In some non-limiting examples, product 100 is applied to a disposable surgical facemask, encapsulated in the delivery system 160 of microcapsules 121 embedded in the facemask. In particular, delivery system 160 comprising microcapsules 121 is applied to a sanitized, middle layer of a 3-layered facemask prior to use of the facemask. Once the user desires to use the new facemask, however, the user must apply force to the facemask, for example by folding and rubbing the sides together to break microcapsules 121 to deliver product 100 to the middle layer of the facemask. Once released from broken microcapsules 121, product 100 dries and thereafter functions as a microscopic bed of product 100 that effectively destroys microbes which encounter the middle layer of the facemask via air currents created by the wearer's breathing. As described herein, delivery system 160, may be utilized with any textile, fabric, cloth, material, object, entity, and/or thing, whether porous or non-porous, to timely deliver product 100 thereto for effective use as an antimicrobial agent.

Embodiments of the delivery system 160 of product 100 may further comprise application of microcapsules 121 encasing product 100 into medical devices and/or medical supplies. In particular, product 100 may be applied via microcapsules 121 to surfaces 140 of bandages, gauze, gauze dressings, sutures, cotton balls, cotton dressings, cotton swabs, cotton tipped applicators, cotton rolls, and other similarly-purposed medical supplies.

Embodiments of the delivery system 160, in some embodiments, further comprise fragrance 130. Fragrance 130 may be encapsulated within microcapsule 121 along with product 100. Fragrance 130 may also be bonded to the exterior surface of microcapsule 121 and configured to react with product 100, upon release of product 100 following rupture of microcapsule 121. Fragrance 130, in some embodiments, is configured to exert, exhibit, or otherwise create a detectable scent for a predetermined period of time following activation of fragrance 130. Embodiments of delivery system 160 may comprise fragrance 130 bearing a scent that persists as long as product 100 remains biologically active. In other words, some embodiments of delivery system 160 comprise a set length of time calculated and configured to match the expected biologically active life of product 100. The biologically active life of product 100 may be accurately determined based upon factors such as the characteristics of substrate 140, external environmental conditions, and concentration and amount of product 100 used, for example. In this manner, the user is able to determine by the presence of the scent emanating from fragrance 130 whether product 100 remains biologically active or has expired. Once expired, the user may determine by the lack of a scent that product 100 on is no longer biologically active and that consideration should be given to sterilize and re-treat or to discard article 142.

Some embodiments of delivery system 160 for product 100 further comprise aging indicator 125. Aging indicator 125, in some embodiments, is encapsulated in microcapsule 121 along with product 100. Aging indicator 125, in some embodiments, is bonded to the exterior surface of microcapsule 121 and configured to activate a coloring agent or fading agent upon contact with product 100. Aging indicator 125, in some embodiments, is configured to exert, exhibit, or otherwise release a color, fading agent, or time-dependent color that changes color over a predetermined period of time after aging indicator 125 has been activated. Some embodiments of delivery system 160 comprise aging indicator 125 comprising a fading color or time-dependent color that changes color or alters color for a time period matching the useful life of product 100's biological activity. In other words, some embodiments of the delivery system 160 comprise a time period calculated and configured to match the anticipated life expectancy of product 100. The biologically active life of product 100 may be determined based upon factors such as the characteristics of surface 140, external environmental conditions, concentration, and amount of product 100 used, for example. In this way, the user is able to determine by the color, faded color, or time-dependent color whether product 100 remains biologically active or has expired. Once expired, the user may determine by the lack of a scent that product 100 on is no longer biologically active and that consideration should be given to re-treat or discard article 142.

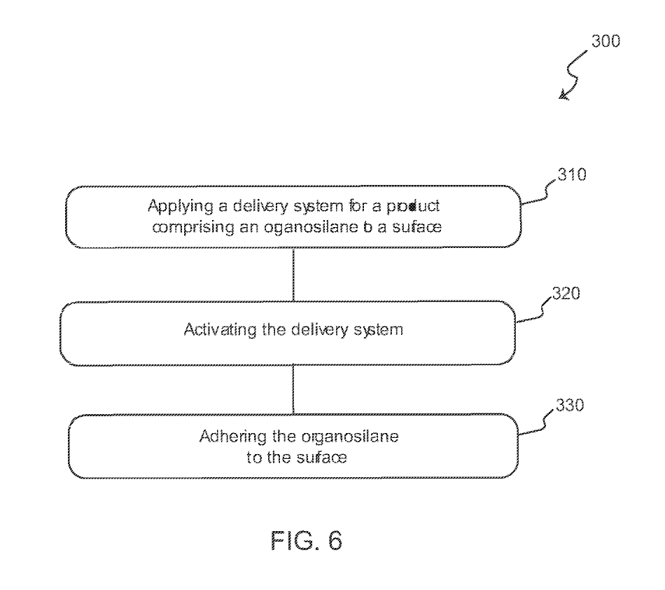

FIG. 6 shows a method of treating infection and/or infectious disease. Applying step 310 of method 300 comprises applying a delivery system for a product comprising an organosilane to a substrate. In some embodiments the delivery system of applying step 310 comprises a microcapsular delivery system, such as microcapsule 121 discussed herein above. In some embodiments, the delivery system of applying step 310 is a delivery system not comprising microcapsules such as by drops or spraying the area to be treated. In some embodiments, applying step 310 comprises integrating the delivery system, such as by imbedding microcapsules containing a product comprising an organosilane for example, into part or all of the material composition of the treated article during manufacture. For example, the felt cuff encircling a portion of a trans-cutaneous central venous catheter, in some embodiments, is manufactured to contain a quantity of microcapsules upon and intermingled within the fibers throughout the felt cuff Activating step 320 of method 300 comprises activating the delivery system. In some embodiments, activating step 320 comprises breaking microcapsules encasing the product comprising the organosilane. An additional non-limiting example of activating step 320, in some embodiments, includes removing the treated article from its packaging. In the central venous catheter example mentioned earlier, activating step 320 may, in some embodiments, comprise passing the segment of the catheter bearing the treated felt cuff tangentially through the skin insertion/puncture site wherein microcapsules encasing product are mechanically ruptured as the cuff passes through the skin. Adhering step 330 of method 300 comprises adhering the organosilane to the substrate. In some embodiments, such as the aforementioned central venous catheter example, adhering step 330 involves reaction of released, activated components of the product, including the organosilane, for example, bonding to the material of the felt cuff, the surface of the catheter, and proteins located within the intercellular matrix of the subcutaneous tissue. Bonding of the organosilane to the surface may be by covalent bonding, ionic bonding, electrostatic bonding, or other interaction between the organosilane and the surface material.

Method 300 may further comprise placing the one or more treated articles into a location prone to microbial growth, such as an area where microbes are known or expected to exist, and/or an area 24 where biofilms have formed or may develop. Alternatively, the treated article may be placed into close proximity to such an area but not directly in the area. By placing the treated article into close proximity but not directly into contact with such an area, the electrostatic properties of product 100 comprising an organosilane or and/or additional cationic detergent or other substance may attract and draw nearby microbes to the cationic product, thereby reducing the concentration of microbes in the adjacent area sought to be protected from microbial colonization and/or infection and possible biofilm formation.

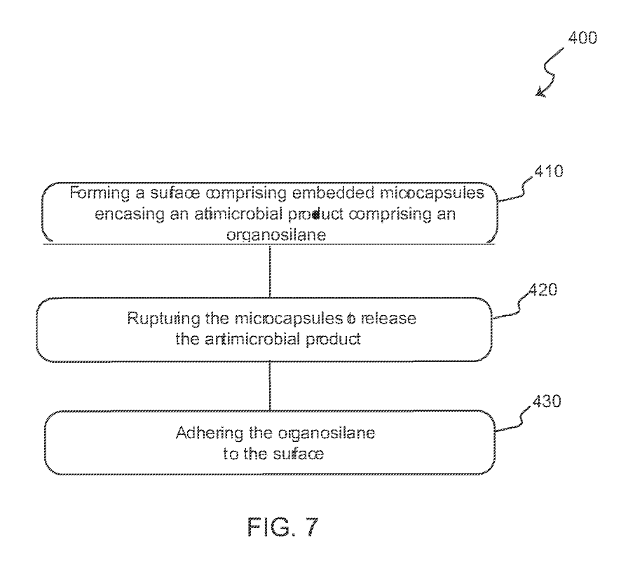

FIG. 7 shows a method 400 of forming an article with an antimicrobial product delivery system embedded therein. Method 400 comprises a forming step 410, a rupturing step 420 and an adhering step 430. Forming step 410, in some embodiments, comprises incorporating a microcapsular delivery system of an antimicrobial product comprising an organosilane into the material composition of an article during manufacture of the article. Rupturing step 420 comprises rupturing of the microcapsules by physical/mechanical, chemical, or electrical means, thereby effecting the release of the antimicrobial product from the delivery system. Adhering step 430 comprises adherence of the organosilane molecules to the surface, such as by covalent bonds, ionic bonds, electrostatic forces, or other molecular interactions between the organosilane and the material composition of the treated surface.

The disclosed methods of application minimize leaching of antimicrobial into the environment, minimizes opportunities for development of microbial resistance due to its combined mechanical and electrostatic mechanisms of action, is safe and effective for use to filter air borne pathogens to and from the respiratory system, in dressings to aid in treating infections and may be incorporated directly onto articles such as medical devices and supplies by way of a delivery system.

Referring again to FIG. 6, depicted is a method of treating infection and/or infectious disease. Applying step 310 of method 300 comprises applying a delivery system for a product comprising an organosilane to a substrate. In some embodiments, the delivery system of applying step 310 comprises a microcapsular delivery system, such as microcapsule 121 discussed herein above. In some embodiments, the delivery system of applying step 310 is a delivery system not comprising microcapsules such as by drops or spraying the area to be treated. Activating step 320 of method 300 comprises activating the delivery system. In some embodiments, activating step 320 comprises breaking microcapsules encasing the product comprising the organosilane. In some embodiments, breaking microcapsules occurs when a dog or other animal scratches the treated substrate in response to itching caused by inflammation. Adhering step 340 of method 300 comprises adhering the organosilane to the substrate. In some embodiments, adhering step 340 involves reaction of released, activated components of the product, such as the organosilane for example, to the material composition of the substrate. For example, the organosilane adheres to protein molecules such as keratin, collagen, and other proteins located on the skin surface or within the intercellular matrix of subcutaneous and deeper tissue. Bonding of the organosilane to the animal tissue substrate is ordinarily by covalent bonding, but can be due to ionic bonding, electrostatic bonding, or other interaction.

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above.

What is claimed is:

1. A method to treat a biofilm present in the ear canal of an animal, comprising: applying an antimicrobial product comprising an aqueous solution of 3-(trihydroxysilyl)propyldimethyloctadecyl ammonium chloride to the ear canal of the animal, wherein the aqueous solution does not contain methanol; and repeating the application of the antimicrobial product to prevent drying of the ear canal.

2. The method of claim 1, wherein the antimicrobial product is applied once daily.

3. The method of claim 1, wherein the antimicrobial product is applied twice daily.

4. The method of claim 1, wherein the animal has bacterial otitis externa.

5. The method of claim 1, wherein the animal has fungal otitis externa.

6. The method of claim 1, wherein the animal has bacterial/fungal otitis externa.

7. The method of claim 1, wherein the antimicrobial product further comprises an anti-inflammatory compound.

8. The method of claim 7, wherein the anti-inflammatory compound is selected from triamcinolone diacetate, hydrocortisone, beta methasone valerate, and beta methasone diproprionate.

9. The method of claim 1, wherein the antimicrobial product further comprises a topical anesthetic.

10. The method of claim 9, wherein the topical anesthetic is selected from lidocaine hydrochloride, benzocaine hydrochloride, hexylresorcinol, and methyl resorcinol.

11. The method of claim 1, wherein the antimicrobial product is applied using a dropper.

12. The method of claim 1, wherein the antimicrobial product is applied as an aerosol.

13. The method of claim 1, wherein the biofilm leads to an ear infection in the animal.

14. The method of claim 13, wherein the antimicrobial product further comprises a proteolytic hydrolase enzyme.
